(19) United States
(12) Patent Application Publication
(10) Pub. No.: US 2026/0301175 A1
(43) Pub. Date: Oct. 1, 2026

(54) MULTI-MODAL METHODS FOR GRADING AN ARTERIOVENOUS MALFORMATION AND SYSTEMS THEREOF

(71) Applicant: Siemens Healthineers AG, Forchheim (DE)

(72) Inventors: Mohammad ABDISHEKTAEI, Charlottesville, VA (US); Varghese KOLLERATHU, Whitefield, Bengalurur (IN); Rahul RAMESH, Avalahalli (IN); Abhinandan TEJANI, Kudlu (IN); Vineet VINAY BHOMBORE, Bengaluru (IN); Matthias WOLF, Coatesville, PA (US); Halid YEREBAKAN, Carmel, IN (US)

(73) Assignee: Siemens Healthineers AG, Forchheim (DE)

(21) Appl. No.: 19/633,106

(22) Filed: Mar. 30, 2026

(30) Foreign Application Priority Data
Mar. 31, 2025 (EP) 25167362.0

(51) Int. Cl. G06T 7/00; G06T 7/60; G16H 20/40; G16H 50/70

(52) U.S. Cl. CPC G06T 7/0012; G06T 7/60; G16H 20/40; G16H 50/70; G06T 2207/10116; G06T 2207/20081; G06T 2207/20084; G06T 2207/30016; G06T 2207/30096; G06T 2207/30101

(57) ABSTRACT

One or more example embodiments relates to a computer-implemented method for characterizing a lesion in a blood vessel of a brain depicted in a radiography image comprising identifying a void, in the radiography image, wherein the void is representative of a presence of the lesion, determining a distance from the identified void to a region of interest in the radiography image, determining a size of a nidus in the radiography image, determining a presence of a deep venous drainage in the radiography image and determining a grading of the lesion based on at least one of the determined distance of the void to the region of interest, the size of the nidus or the presence of the deep venous drainage.

### CROSS-REFERENCE TO RELATED APPLICATION(S)

The present application claims priority under 35 U.S.C. § 119 to European Patent Application No. 25167362.0, filed Mar. 31, 2025, the entire contents of which is incorporated herein by reference.

### FIELD

One or more example embodiments relates to a computer-implemented method, a computer-implemented apparatus, a system and a computer-program product for characterizing a lesion in a blood vessel of a brain depicted in a radiography image.

### RELATED ART

Medical imaging has provided a powerful tool for diagnosing abnormalities in a body of a patient for many years and it has developed to represent a standard procedure in state-of-the-art medical diagnostics. Medical imaging made it possible to support a non-invasive diagnosis based on images that were captured of certain parts of a body of a patient. Said images were, e.g., captured via X-ray imaging, computed tomography (CT) imaging, positron emission tomography (PET) imaging, ultrasonic imaging, etc.

The imaging quality of these imaging methods has drastically improved over the last decades supporting the accurate identification of ever decreasing abnormalities. The imaging quality has further improved such that it allows precise capturing of, e.g., X-rays which have penetrated the body of the patient with an improved contrast ratio (as compared to respective devices used decades ago) and suppressed blurring effects. These improvements in the imaging quality have advantageously contributed to a general improvement of medical imaging and has led to an increase in the constant demand for medical imaging even further.

However, even nowadays, the acquired radiography images (such as, e.g., magnetic resonance imaging (MRI)) are, to a wide extent, still analyzed manually by an experienced physician. This is time and resource intensive and leads to high costs of medical imaging which, in turn, need to be compensated by already weakened healthcare systems and are accompanied by prolonged waiting times for the patients requiring a medical imaging-based diagnosis.

The detection of small anomalies in the body of a patient plays a dominant role when small blood vessels need to be investigated. These small-scale structures can oftentimes not be reliably identified based on currently existing (mostly manual) methods of reviewing medical images. However, even though some blood vessels may be small, they may nevertheless be subject to severe and threatening diseases or may even be their origin.

This may be of relevance when lesions of the blood vessels are formed, leading to arteriovenous malformations (AVMs) formed by a group of blood vessels. In such malformations, arteries and veins may unusually be tangled and may form direct connections, bypassing normal blood vessels.

AVM is a neurovascular disorder wherein there is an absence of capillaries in blood vessels. This may lead to a low resistance pathway for the blood stream. AVMs are the second largest cause of cerebral hemorrhage. Children tend to be more susceptible to cerebral hemorrhage and the associated mortality rate is 10-15%.

The tangled formations may also be referred to as a nidus of an AVM. The identification of such a nidus may be seen as crucial to prevent a deterioration of the health state of a patient which may arise from the presence of the nidus.

What is more, even if a nidus is detected, a decision to be made by a medical practitioner is whether to treat the underlying AVM or not. However, up to today, a demarcation of arteries and veins in respective radiography images is mainly done manually. This may require reviewing a plurality of angiography images, identifying arteries and veins manually and deciding based thereon, whether a nidus (e.g., of harmful size) is present or not.

Even if it is determined that a lesion (e.g., based on a nidus) is present in a radiography image, it still requires an additional manual step to decide whether the lesion is to be treated (e.g., by surgery) or not.

In a clinical setting, when it is required to decide whether a lesion is to be treated, medical practitioners may make use of guidelines and pre-defined grading systems such as a Spetzler-Martin grading system for quantifying a lesion. For example, patients with lower grades (I and II) according to the Spetzler-Martin grading system may represent suitable surgical candidates, as they may have a lower risk of surgery-related neurological deficits. In contrast, patients with higher grades (IV and V) according to the Spetzler-Martin grading system may be at a high risk of surgical complications and may usually be managed conservatively or with non-surgical methods like stereotactic radiosurgery or embolization.

### SUMMARY

there is still a need to improve the analysis of radiography images, in particular, when it comes to the analysis of small-scale structures, such as, e.g., blood vessels, which is currently not yet fulfilled in a satisfying manner based on the presently known and applied procedures.

Therefore, one or more example embodiments improve the characterization of lesions.

### BRIEF DESCRIPTION OF THE DRAWINGS

Embodiments, features and advantages will become apparent from the subsequent description and dependent claims, taken in conjunction with the accompanying drawings, in which:

FIG. 1 depicts an exemplary method for identifying a void;

FIG. 2 depicts an exemplary method for determining a distance;

FIGS. 3A and 3B depict an exemplary method for differentiating feed arteries from draining veins;

FIG. 4 depicts an exemplary method for semi-automatically segmenting arteries;

FIG. 5 depicts an exemplary method for determining a size of a nidus;

FIGS. 6A and 6B depict an exemplary method for determining a presence of a deep venous drainage;

FIG. 7 depicts an exemplary method for determining a grading of the lesion;

FIG. 8 depicts an exemplary method for suggesting a treatment of a lesion;

FIG. 9 depicts a workflow of a computer-implemented method for characterizing a lesion in a blood vessel;

FIG. 10 depicts an exemplary computer-implemented apparatus for characterizing a lesion in a blood vessel; and

FIG. 11 depicts an exemplary system for characterizing a lesion in a blood vessel.

### DETAILED DESCRIPTION

According to one or more example embodiments, a computer-implemented method for characterizing a lesion in a blood vessel of a brain depicted in a radiography image is suggested. The computer-implemented method may comprise identifying a void in the radiography image, wherein the void is representative for a presence of the lesion, determining a distance of the identified void to a region of interest in the radiography image and determining a size of a nidus in the radiography image. Moreover, the computer-implemented method may comprise determining a presence of a deep venous drainage in the radiography image and determining a grading of the lesion based on the determined distance of the void to the region of interest, based on the size of the nidus and/or based on the determined presence of the deep venous drainage.

In some examples, the void may be associated with (e.g., may represent) a location of the lesion.

By determining the grading of the lesion based on the determined distance of the void to the region of interest, based on the size of the nidus and/or based on the determined presence of the deep venous drainage, a multi-modal computer-implemented (and thus automated) method may be provided to efficiently characterize a lesion in a blood vessel of a brain of a patient. This may generally support a faster characterization of the lesion and may support a more accurate diagnosis of the respective lesion as well as a decision whether to treat (e.g., by surgery) or not. Moreover, a multi-modal characterization of the lesion may be provided as more than one parameter may be considered when determining the grading.

In an embodiment, the identifying of the void may comprise identifying the void based on a trained artificial intelligence model, preferably by a trained classification model, more preferably by a Residual Network, ResNet.

In case no void is detected in the radiography image, it may be concluded that no lesion is present in the radiography image. In such a case, the computer-implemented method may be terminated. In some examples, a standard value (e.g., a logical 0 or 1) may be output by the computer-implemented method, indicating that no void was detected in the radiography image.

A classification model may be referred to as a type of artificial intelligence model designed to categorize or group data into predefined classes or categories based on specific features or characteristics. A respective classification model may learn patterns and relationships from labeled training data, where each data point may be associated with a known class. Once trained, classification models may predict the class of new, unseen data points by analyzing their features and applying the learned patterns.

Classification models may be configured to handle different types of classification tasks, including binary classification (where data is sorted into two classes) and multi-class classification (involving more than two classes). Prior to applying a classification model for the actual classification task, preceding training of the model on a labeled dataset may occur, evaluating its performance on test data, and then applying it to real-world scenarios to classify new, unseen instances.

A ResNet may be referred to as a deep learning artificial intelligence model designed to address the challenges of training (deep) artificial intelligence models, such as neural networks. It may be based on the concept of residual learning, where each layer of the deep neural network may learn residual functions with reference to the layer inputs, rather than learning unreferenced functions. This approach may allow for the creation of much deeper networks without suffering from a vanishing gradient problem. This may be facilitated by incorporating skip connections, also known as shortcut connections, which may enable the network to bypass one or more layers. These connections may facilitate the flow of information across the neural network, making it easier to train and optimize very deep models.

This may generally support an automated, accurate and thus efficient identifying of a void in the radiography image under investigation.

In another embodiment, the region of interest may be an eloquent area of the brain.

An eloquent area of the brain may be referred to an area that is responsible for functions of the patient like movement, sensation and/or speech.

By determining a distance from the void to the region of interest, a risk indicator may be derived which may indicate a risk for treating the void by surgery.

In another embodiment, the determining of the distance may comprise mapping an atlas of the brain to the radiography image, providing the mapped radiography image to a trained artificial intelligence model which is trained for localizing a lesion in a radiography image and localizing the lesion in the radiography image, based on the respectively trained artificial intelligence model, wherein the localizing preferably comprises determining coordinates of the lesion.

A lesion may be referred to as an anomaly in a tissue depicted in the radiography image.

The mapped radiography image may be provided with an atlas-based annotation of anatomical regions/nuclei of the brain of the patient.

In some examples, the atlas may represent anatomical regions of the (human) brain. In some examples, the atlas may be a Morel atlas.

The coordinates may indicate the location of the lesion in a cartesian coordinate system, e.g., in a two-dimensional coordinate system (X-Y-coordinate system) or in a three-dimensional coordinate system (X-Y-Z-coordinate system).

In some examples, the artificial intelligence model, trained for localizing the lesion, may be a convolutional neural network (CNN), e.g., comprised by a U-net architecture.

The coordinates may indicate a center of the lesion. The center may be a point comprised by the lesion that has a same distance to each point of a demarcation line surrounding the lesion.

In another embodiment, the mapping of the atlas may comprise a rigid mapping.

A rigid mapping may be referred to as a type of transformation that preserves the shape and size of an object, only allowing for changes in position and orientation. The rigid mapping may be used to align or transform information from the atlas while maintaining its fundamental structure. Further, beside translation and rotation, scaling may be used, particularly a similarity transformation. Thus, in embodiments, a non-rigid transformation may be used, being adapted to provide accurate results.

In some examples, it may also be possible that the mapping is non-rigid.

In another embodiment, the determining of the size of the nidus may comprise identifying an artery and/or a vein in the radiography image and determining a location of the nidus in between the identified artery and the identified vein. In particular, in some situations, contrast may be injected and there are phases where arteries are more visible than veins and vice-versa. Moreover, the location of the void may imply the location of the nidus. The size of nidus has more clinical relevance. The location of the lesion may be used to determine proximity to eloquent areas.

In some examples, the radiography image may be provided as a digital subtraction angiography, DSA, image.

In some examples, a raw radiography image and a segmented image (e.g., a radiography image onto which segments of the atlas are mapped) may be retrieved from a same or different remote entities (e.g., through a secure channel). In particular, a secure channel may include whitelisted IP address enabling the transfer of data.

By determining the location of the nidus, a location of a potential lesion may be further specified based on which a subsequent grading of the lesion may be performed.

In another embodiment, the lesion may be an arteriovenous malformation, AVM.

An AVM may be referred to as a congenital vascular abnormality characterized by a tangled network of blood vessels where arteries connect directly to veins (e.g., arising from a formation of a nidus) without the usual intervening capillary bed. This abnormal connection may create a high-pressure, high-flow system that bypasses normal tissue oxygenation. The absence of capillaries in the malformation can lead to weakened blood vessel walls, increasing the risk of rupture and subsequent hemorrhage.

Via the computer-implemented method set out herein, an improved and automated characterization of an AVM may be facilitated.

In another embodiment, the computer-implemented method may further comprise associating the lesion, based on the determined size of the nidus, with a grade of a grading system, preferably of a Spetzler-Martin grading system, wherein the grading system indicates a suitability of the lesion and the patient for a treatment of the lesion by surgery.

In some examples, the grading system may comprise five grades, wherein grade I may be associated with a minimal surgical risk and wherein grade V may be associated with a very high surgical risk, often considered as inoperable.

In some examples, the Spetzler-Martin grading system may be referred to as a classification system for quantifying a surgical risk of AVMs.

This may enable an accurate and automized determination, whether a lesion may be treated by surgery or not.

In another embodiment, the computer-implemented method may further comprise suggesting a treatment for the lesion based at least in part on the determined grading of the lesion and preferably based at least in part on one or more of an age of a patient, country specific guidelines for treating the lesion and/or a parameter that is used to determine the grading.

The country specific guidelines may refer to a recommended treatment procedure for treating the lesion in case certain preconditions (e.g., the determined grading of the lesion) are present.

The parameter that is used to determine the grading may be one or more of the determined distance of the void to the region of interest, based on the size of the nidus and/or based on the determined presence of the deep venous drainage.

In another embodiment, the suggesting of the treatment may comprise querying a database for patients with a similar grading and their respective administered treatment.

A grading may be referred to as similar if the grading differs from the grading, determined by the computer-implemented method as set out herein, by +/−1 grading step, e.g., on a grading scale which spans across grading steps from 0 to 5.

An administered treatment may be a treatment that has historically been assigned to a patient with a respective grading step.

This may support an improved decision making when suggesting a treatment for a lesion of a blood vessel of a patient. This may be facilitated as historic administered treatments and their potential outcomes may be taken into consideration when suggesting the treatment for the patient at the present time.

In another embodiment, the computer-implemented method may further comprise storing the suggested treatment to a database.

In some examples, the suggested treatment may be stored together with the determined grade.

The database may be located at a remote entity, i.e., an entity that is spatially different from the entity that is configured to execute the computer-implemented method as set out herein.

This may efficiently support an increase of the dataset stored in the database and may thus support and improve a subsequent and tailored suggesting of a treatment for an identified lesion.

According to a second aspect, a computer program product is suggested. The computer program product may comprise instructions which, when the program is executed by a computer, cause the computer to carry out the computer-implemented method as set out above.

A computer program product, such as a computer program means, may be embodied as a memory card, USB stick, CD-ROM, DVD or as a file which may be downloaded from a server in a network. For example, such a file may be provided by transferring the file comprising the computer program product from a wireless communication network.

According to a third aspect, a computer-implemented apparatus for characterizing a lesion in a blood vessel depicted in a radiography image is suggested. The computer-implemented apparatus may comprise an identifying unit configured for identifying a void, in the radiography image, that is representative for a presence of the lesion, a determining unit configured for determining a distance of the identified void to a region of interest in the radiography image, a determining unit configured for determining a size of a nidus located in between the identified artery and the vein, a determining unit configured for determining a presence of a deep venous drainage in the radiography image and a determining unit configured for determining a grading of the lesion based on the determined distance of the void to the region of interest, based on the size of the nidus and/or based on the determined presence of the deep venous drainage.

According to an aspect, the computer-implemented apparatus may further comprise an execution unit for executing the computer-implemented method as set out above.

Each of the units mentioned herein may be provided as or may comprise a respective processor (e.g., as a central processing unit (CPU)) and/or a field programmable gate array (FPGA)) and/or a graphics processing unit (GPU) and/or a tensor processing unit (TPU). In some examples, the respective units may comprise a memory.

According to a fourth aspect, a system for characterizing a lesion in a blood vessel depicted in a radiography image is suggested. The system may comprise the computer-implemented apparatus as set out above and the computer-program product as set out above.

Even though some embodiments are described in isolation from each other, it is emphasized that the embodiments may nevertheless be combined with each other.

The embodiments and features described with reference to an apparatus according to one or more example embodiments, mutatis mutandis, to a method according to one or more example embodiments.

Further possible implementations or alternative solutions of the invention also encompass combinations-that are not explicitly mentioned herein-of features described above or below with regard to the embodiments.

In the Figures, like reference numerals designate like or functionally equivalent elements, unless otherwise indicated.

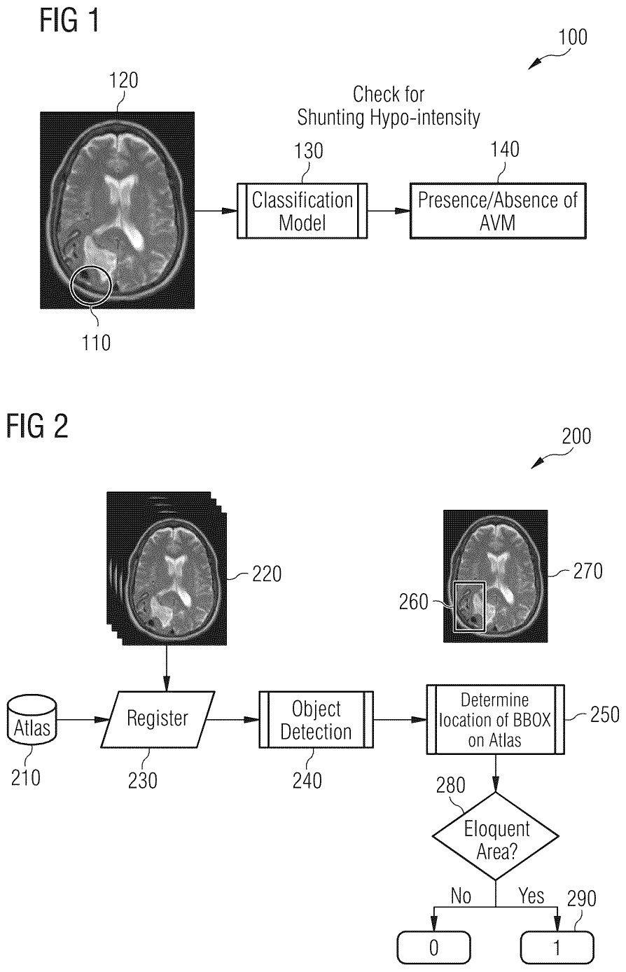

FIG. 1 depicts an exemplary method 100 for identifying a void 110 in a radiography image 120, e.g., an MRI.

The radiography image 120 may be provided to a trained artificial intelligence model 130, which may preferably be implemented as a classification model. The trained artificial intelligence model 130 may have been trained to identify a void in the radiography image 120.

An output of the trained artificial intelligence model 130 may indicate a presence/absence of AVM 140 in the radiography image 120. In this regard, it may be noted that a void in T2 MRI depicts a low resistance pathway for the blood to flow. In particular, not all voids mean AVMs. But, the first AI model (ResNet) based one may be trained to classify voids arising from AVMs as positive class. The model could be modified to output three classes namely, “void due to AVM”, “void due to other pathological condition”, and “no void”.

In some examples, the presence of the AVM may be indicated by the trained artificial intelligence model 130 by outputting a logical 1 (or any other suitable indicator). In some examples, the absence of the AVM may be indicated by the trained artificial intelligence model 130 by outputting a logical 0 (or any other suitable indicator).

In case the lesion is an AVM, AVMs may oftentimes lack capillary components that may lead to arterialized veins in a high-flow, low-resistance shunt system. A fast blood flow may generate flow voids and may thus be seen on T2 weighted magnetic resonance images which are generally hypo-intense in nature.

In MRI, hypo-intense may refer to areas or tissues that appear darker compared to surrounding tissues on an image. This lower signal intensity is relative to the adjacent structures and can vary depending on the specific MRI sequence used. In some examples, on T2-weighted images, hypo-intense regions can suggest certain tumors, dense tissue such as calcified lesions, or iron deposits.

FIG. 2 depicts an exemplary method 200 for determining a distance of the identified void (e.g., according to the method 100) to a region of interest in the radiography image.

In some examples, an atlas 210 may be provided. An atlas may be referred to as a standardized reference framework that maps, names and delineates various anatomical and functional regions within, e.g., the brain of the patient.

Subsequently, a plurality of radiography images 220 (e.g., MRI) may be provided and registered 230. In some cases, only a single radiography image 220 may be provided and registered 230. In particular, registering may include transforming two images into one coordinate system.

Subsequently, an object detection 240 may be executed. The object detection 240 may be configured to determine a plurality of objects such as anatomical regions in the provided plurality of radiography images 220. The determining may, e.g., comprise identifying the void in the provided plurality of radiography images 220. In some examples, the determining of the plurality of objects may further comprise a determining of respective locations of the determined plurality of anatomical regions.

In an example, as part of the object detection 240, a box 260 may be drawn about a determined object in a radiography image 270 to highlight the respective determined object.

Subsequently, the objects determined as part of the object detection 240 (as well as their location) may be compared 250 to the atlas 210. The comparing may comprise a mapping of anatomical regions represented in the atlas 210 onto the objects determined as part of the object detection 240. In other words, the objects determined as part of the objection detection 240 may be associated with respective anatomical regions set out in the atlas 210. This may effectively lead to an annotation and labeling, respectively, of the objects determined as part of the object detection 240 and of the object, highlighted by box 260.

Based on the mapping, a determining 280 is performed, whether an object determined as part of the object detection 240 lies in area region of interest such as, e.g., an eloquent area.

The result of the determining 280, whether an object determined as part of the object detection 240 lies in a region of interest or not is output 290 (e.g., on a screen and/or as a flag and/or as a logical number which may subsequently be further processed).

In case the result of the determining 280 is output 290 as a logical number, a logical 1 may be output if it is determined 280 that the object lies in a region of interest. Alternatively, a logical 0 may be output if it is determined 280 that the object does not lie in a region of interest.

In case it is determined that the object lies in a region of interest (or is distanced from a region of interest by a metric separation that lies below a predetermined threshold), a treatment of the lesion may be suggested that is non-invasive to keep a potential risk for the patient as low as possible.

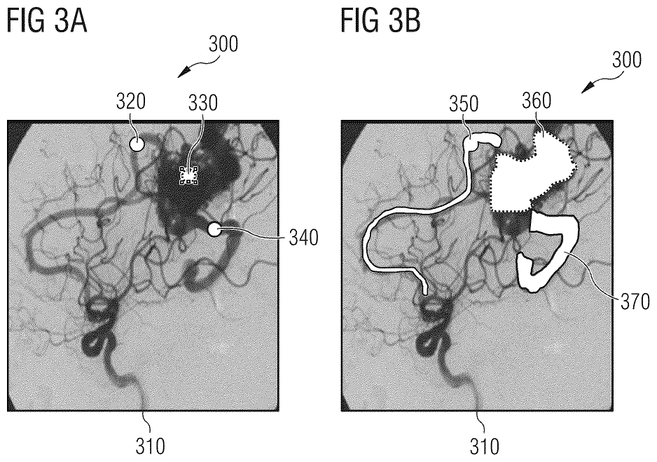

FIGS. 3A and 3B depict an exemplary method 300 for differentiating feed arteries from draining veins in a radiography image 310.

More specifically, FIG. 3A depicts an exemplary radiography image 310 superimposed by a user input that indicates reference points 320, 330 and 340 which reference points 320, 330 and 340, in turn, associate structures of the radiography image 310 to arteries/veins and a nidus depicted in the radiography image 310.

In some examples, a single reference point is provided for each artery, vein and nidus depicted in the radiography image 310. In some examples, more than one reference point may be provided for each artery, vein and nidus depicted in the radiography image 310.

FIG. 3B depicts the exemplary radiography image 310 of FIG. 3A, wherein the artery, vein and the nidus depicted thereon has been determined (i.e., identified) and highlighted.

Based on the user input (i.e., based on the reference points 320, 330 and 340), the arteries 350, veins 370 and the nidus 360 may be determined and highlighted, e.g., by a trained artificial intelligence model that has been trained to determine arteries, veins and/or a nidus in a radiography image based on initially provided reference points.

In some examples, the respective highlighting may comprise a contouring (e.g., drawing a demarcation line) of the arteries, veins and the nidus. In some examples, the highlighting may comprise a filling of the respective arteries, veins and the nidus with color.

This may effectively provide a semi-automatic segmentation procedure for determining arteries, veins and/or a nidus based on an initially provided user input such as providing the reference points 320, 330 and 340.

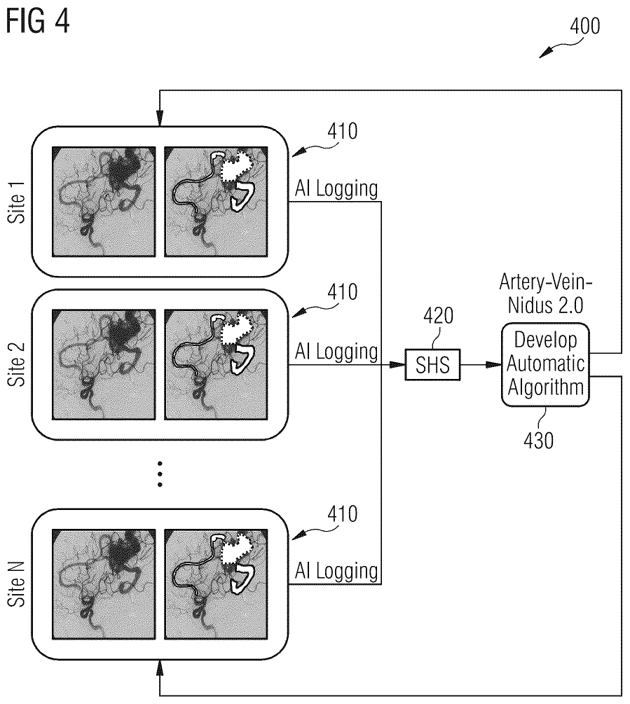

FIG. 4 depicts an exemplary method 400 for semi-automatically segmenting arteries, veins and a nidus in a radiography image, e.g., the radiography image 310.

As outlined with respect to FIGS. 3A-3B, above, the segmentation may be based on reference points provided, e.g., by a user (e.g., medical practitioner), that indicate a location in a radiography image which depicts an artery, a vein and/or a nidus.

The exemplary procedure 400 may be based on retrieving a plurality of historically associated pairs of reference points and arteries, veins and/or a nidus 410, e.g., from a remote entity. The plurality of historically associated pairs of reference points and arteries, veins and/or a nidus 410 may comprise pairs of images wherein a respective pair comprises a radiography image with reference points and an image with a respectively segmented artery, vein and/or a nidus based on the reference points.

In some examples, the retrieving may comprise an logging procedure to collect interaction, such as, e.g., click points.

The plurality of historically associated pairs of reference points and arteries, veins and/or a nidus 410 may be retrieved through a secure channel 420.

The pairs of reference points and arteries, veins and/or a nidus 410 may be retrieved by an algorithm 430. In some examples, the algorithm 430 may be provided as a trained artificial intelligence model which may be trained (based at least in part on the retrieved pairs of reference points and arteries, veins and/or a nidus 410) to determine an anatomical structure of arteries, veins and/or a nidus in a radiography image based on initially provided reference points.

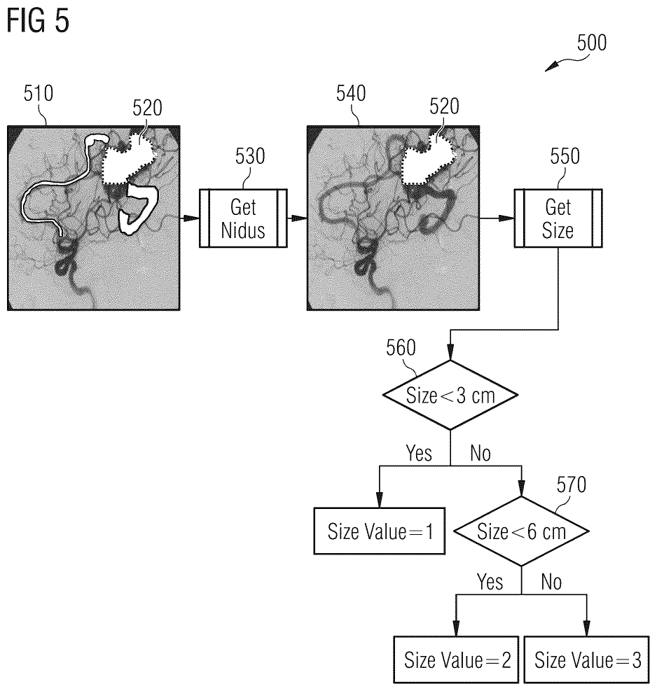

FIG. 5 shows an exemplary method 500 for determining a size of a nidus in a radiography image 510.

The radiography image 510 may be provided. A nidus 520 may have been determined and highlighted in the radiography image 510, e.g., according to at least one of the methods 300 and/or 400 as outlined above.

Subsequently, a nidus segmentation 530 may be performed. The nidus segmentation 530 may delete other anatomical regions in the radiography image 510 which were not determined as a nidus 520 in at least one of the methods 300 and/or 400.

That is, a reduced radiography image 540 may be provided that only depicts the nidus 520 as a highlighted anatomical region.

Subsequently, a size 550 may be derived from the radiography image 540.

Afterwards, it may be determined 560, whether the size of the nidus 520 exceeds a first predefined threshold (e.g., whether the size of the nidus 520 exceeds 3 cm or not). The first predefined threshold may be defined by an underlying grading system, such as, e.g., the Spetzler-Martin grading system (but not limited thereto).

If it is determined that the nidus is smaller than the first predefined threshold, a respective output may be provided (e.g., a 1 or any other suitable number).

If it is determined that the size of the nidus 520 exceeds the first predefined threshold, it may be determined 570 whether the size of the nidus 520 exceeds a second predefined threshold (e.g., whether the size of the nidus 520 exceeds 6 cm or not).

If it is determined that the size of the nidus 520 is smaller than the second predefined threshold, a respective output may be provided (e.g., a 2 or any other suitable number).

If it is determined that the size of the nidus 520 is larger than the second predefined threshold, a respective output may be provided (e.g., a 3 or any other suitable number).

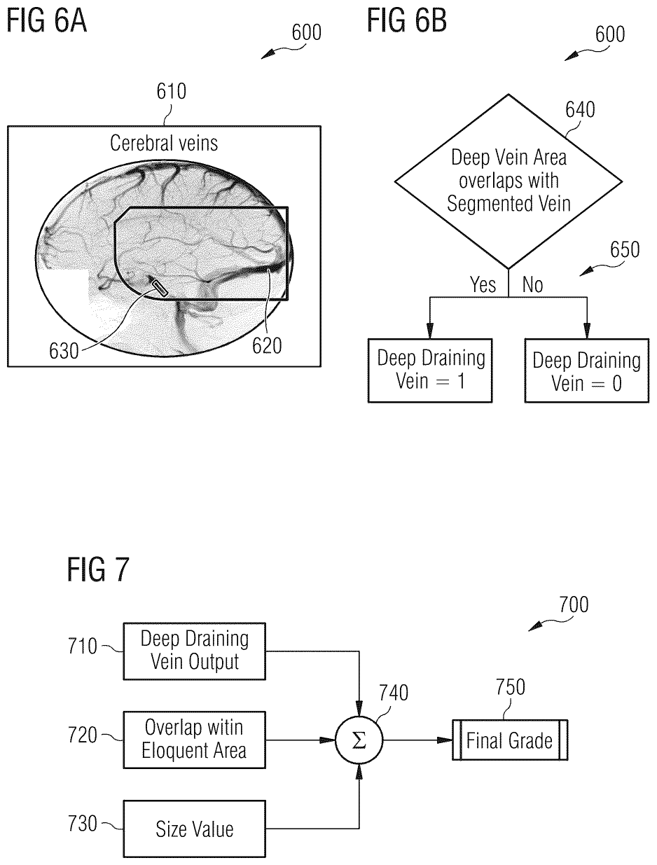

FIGS. 6A and 6B show an exemplary method 600 for determining a presence of a deep venous drainage in a radiography image (e.g., the radiology image 310, 510).

FIG. 6A depicts a radiography image 610 (e.g., a DSA image).

The method 600 may be initiated by providing the radiography image 610, e.g., depicting cerebral veins.

The radiography image 610 may be provided with a highlighted region, indicating a potential deep vein area 620 that indicates a potential region in the radiography image 610 in which the probability that deep veins may be found is higher than in a surrounding region. The potential deep vein area 620 may be provided by a medical practitioner (e.g., a radiologist). The potential deep vein area 620 may be provided manually (e.g., by a medical practitioner).

In some examples, veins (or a single vein) depicted in the radiography image 610 may have been segmented beforehand. In some examples, the veins may have been segmented according to any of the methods 300 and/or 400 as set out with reference to FIGS. 3 and 4, above. In some examples, veins may have been segmented according to another segmentation procedure which is not expressly disclosed herein.

The segmented veins 630 may be overlaid onto the radiography image 610.

In case an overlay arises between the segmented veins 630 and the potential deep vein area 620, the segmented veins 630 are referred to as deep veins.

Deep lying veins may, e.g., be one or more of a sigmoid sinus, a straight sinus and/or a lateral sinus.

Veins such as superior sagittal sinus, superior cerebral veins, sphenoparietal sinus may be referred to as superior veins.

FIG. 6B shows a flowchart illustrating the determining of the presence of the deep venous drainage in the radiography image 610.

At first, it may be determined 640, whether the potential deep vein area 620 overlaps with the segmented veins 630.

Based thereon, an output 650 may be provided.

In some examples, if it is determined that the deep vein area overlaps with the segmented veins, a logical 1 may be output. However, it may also be possible that any other number may be output that indicates an overlap.

In some examples, if it is determined that the deep vein area does not overlap with the segmented veins, a logical 0 may be output. However, it may also be possible that any other number may be output that indicates an absence of an overlap.

FIG. 7 depicts an exemplary method 700 for determining a grading of the lesion.

The method 700 may comprise providing information 710, associated with the presence of a deep venous drainage, to a computation unit 740.

The method 700 may comprise providing information 720, associated with the determining of a distance of the identified void to the region of interest in the radiography image, to the computation unit 740.

The method 700 may comprise providing information 730, associated with the determining of the size of the nidus in the radiography image, to the computation unit 740.

The computation unit 740 may be configured to calculate a grading 750 of the lesion based on one or more the information 710 730 provided to the computation unit 740.

In some examples, the provided information 710-730 may each refer to a number that had been assigned as a result of executing a respective method 200, 500 and/or 600 as set out above. In some examples, the computation unit 740 may be configured to calculate a sum of the numbers provided to the computation unit 740. In some examples, the numbers may be weighted relative to each other.

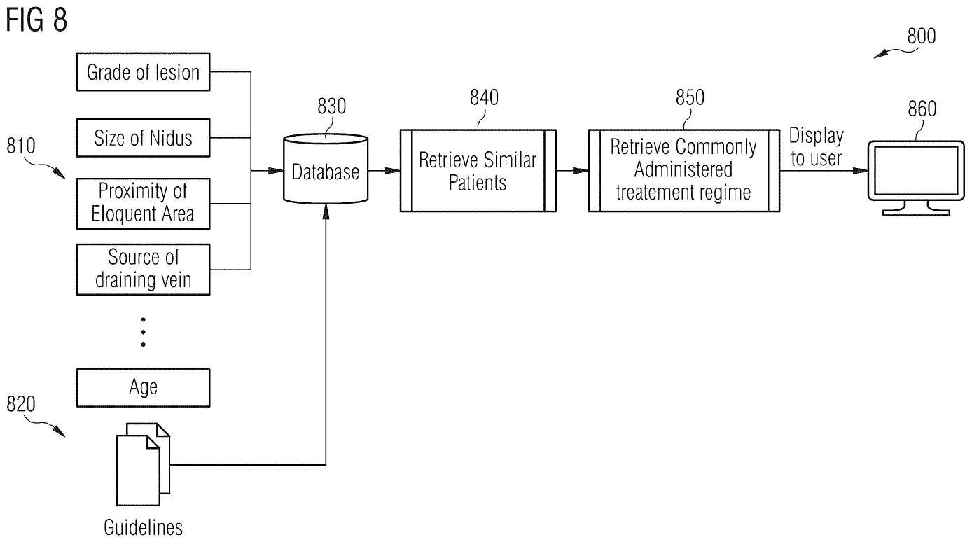

FIG. 8 depicts an exemplary method 800 for suggesting a treatment of a lesion.

The method 800 may comprise providing information associated with the lesion 810 to a database 830. In some examples, the information associated with the lesion 810 may comprise a grade of a lesion, a size of a nidus, a distance between a void and a region of interest, a segmented vein, an age of a patient, etc.

The method 800 may comprise providing (national) guidelines 820 to the database 830.

Based thereon, information associated with similar patients 840 (relative to the patient that is associated with the information provided to the database 830) may be retrieved from the database 830 and may be provided to a medical practitioner.

Based on the information associated with similar patients 840, retrieved from the database 830, a number K, with K≥1, of top treatment protocols may be derived 850 that were administered to the similar patients.

In some examples, said treatment protocols may be provided in a ranked list (e.g., ranked in descending or ascending order with respect to a historically achieved success rate of a respective treatment protocol). In some examples, the ranked list may be ranked according to an expected success rate of a treatment if the treatment is carried out according to a respective treatment protocol. The expected success rate may be calculated by a patient model (e.g., based on a respectively trained artificial intelligence model that may have been trained to determine an excepted success rate of a treatment which may be suggested to treat a lesion).

The resulting suggested treatment (e.g., provided as a treatment protocol) may be displayed to a user 860.

In some examples, it may be possible that the suggested treatment for a patient may be stored to the database 830 (incl. relevant information that was used for determining the suggested treatment). This may improve a quality of information that is stored in the database over the course of time and may make the database 830 growing over time. Due to a growing database, based on which a treatment may be suggested for a patient, the suggesting of a treatment may more closely be tailored to the patient while still being compliant to respective (national) guidelines.

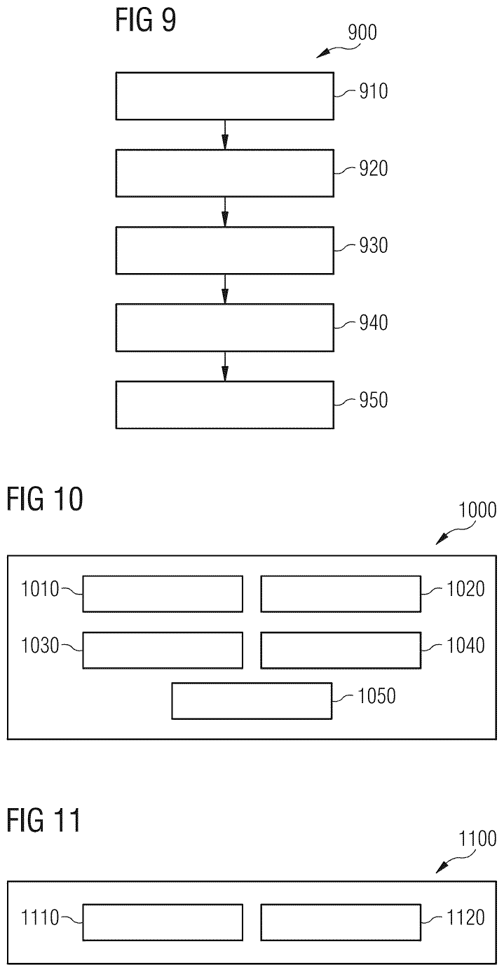

FIG. 9 depicts a workflow of a computer-implemented method 900 for characterizing a lesion in a blood vessel of a brain depicted in a radiography image.

In step 910, a void is identified, in the radiography image, wherein the void is representative for a presence of the lesion.

In step 920, a distance is determined of the identified void to a region of interest in the radiography image.

In step 930, a size of a nidus in the radiography image is determined.

In step 940, a presence of a deep venous drainage in the radiography image is determined.

In step 950, a grading of the lesion is determined based on the determined distance of the void to the region of interest, based on the size of the nidus and/or based on the determined presence of the deep venous drainage.

FIG. 10 shows an exemplary computer-implemented apparatus 1000 for characterizing a lesion in a blood vessel depicted in a radiography image. The apparatus 1000 comprises an identifying unit 1010, a determining unit 1020 for determining a distance, a determining unit 1030 for determining a size, a determining unit 1040 for determining a presence and a determining unit 1050 for determining.

The identifying unit 1010 is configured for identifying a void in the radiography image, that is representative for a presence of the lesion.

The determining unit 1020 is configured for determining a distance of the identified void to a region of interest in the radiography image.

The determining unit 1030 is configured for determining a size of a nidus located in between the identified artery and the vein.

The determining unit 1040 is configured for determining a presence of a deep venous drainage in the radiography image.

The determining unit 1050 is configured for determining a grading of the lesion based on the determined distance of the void to the region of interest, based on the size of the nidus and/or based on the determined presence of the deep venous drainage.

FIG. 11 shows an exemplary system 1100 for characterizing a lesion in a blood vessel depicted in a radiography image.

The system 1100 may comprise a computer-implemented apparatus 1110, e.g., a computer-implemented apparatus as disclosed herein, and a computer-program product 1120 having instructions, e.g., a computer-program product 1120 as described herein. The computer implemented apparatus 1110 may be processing circuitry configured to execute the instructions.

The system 1100 may comprise the computer-program product as disclosed herein.

Although the present invention has been described in accordance with preferred embodiments, it is obvious for the person skilled in the art that modifications are possible in all embodiments.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

### REFERENCE NUMERALS

100 method
110 void
120 radiography image
130 trained artificial intelligence model
140 AVM
200 method
210 atlas
220 radiography image
230 registering step
240 object detection
250 comparison step
260 box
270 radiography image
280 determining step

290 output
300 method
310 radiography image
320 reference point
330 reference point
340 reference point
350 highlighted artery
360 highlighted nidus
370 highlighted vein
400 method
410 historically associated pairs
420 secure channel
430 algorithm
500 method
510 radiography image
520 nidus
530 nidus segmentation
540 radiography image
550 size
560 determination step
570 determination step
600 method
610 radiography image
620 potential deep vein area
630 segmented veins
640 determining step
650 output
700 method
710 information associated with a presence of a deep venous drainage
720 information associated with determining a distance
730 information associated with determining a size
740 computation unit
750 grading
800 method
810 lesion
820 guidelines
830 database
840 information associated with similar patients
850 derived treatment protocols
860 user
900 computer-implemented method
910 step
920 step
930 step
940 step
950 step
1000 computer-implemented apparatus
1010 identifying unit
1020 determining unit for determining a distance
1030 determining unit for determining a size of a nidus
1040 determining unit for determining a presence
1050 determining unit for determining a grading
1100 system
1110 computer-implemented apparatus
1120 computer-program product

1. A computer-implemented method for characterizing a lesion in a blood vessel of a brain depicted in a radiography image, the computer-implemented method comprising:
identifying a void in the radiography image, wherein the void is representative of a presence of the lesion;
determining a distance from the void to a region of interest in the radiography image;
determining a size of a nidus in the radiography image;
determining a presence of a deep venous drainage in the radiography image; and
determining a grading of the lesion based on at least one of the determined distance from the void to the region of interest, size of the nidus or the presence of the deep venous drainage.

2. The computer-implemented method of claim 1, wherein the identifying of the void identifies the void based on a trained artificial intelligence model.

3. The computer-implemented method of claim 1, wherein the region of interest is an eloquent area of the brain.

4. The computer-implemented method of claim 1, wherein the determining the distance comprises:
mapping an atlas of the brain to the radiography image;
providing the mapped radiography image to a trained artificial intelligence model, the trained artificial intelligence model being trained for localizing a lesion in a radiography image; and
localizing the lesion in the radiography image based on the trained artificial intelligence model.

5. The computer-implemented method of claim 4, wherein the mapping of the atlas comprises a rigid mapping.

6. The computer-implemented method of claim 1, wherein the determining the size of the nidus comprises:
identifying an artery and a vein in the radiography image; and
determining a location of the nidus between the identified artery and the identified vein.

7. The computer-implemented method of claim 1, wherein the lesion is an arteriovenous malformation.

8. The computer-implemented method of claim 1, further comprising:
associating the lesion, based on the determined size of the nidus, with a grade of a grading system, wherein the grading system indicates a suitability for the lesion and a patient for a treatment of the lesion by surgery.

9. The computer-implemented method of claim 1, further comprising:
suggesting a treatment for the lesion based at least in part on the determined grading of the lesion.

10. The computer-implemented method of claim 9, wherein the suggesting the treatment comprises:
querying a database for patients with a similar grading and respective administered treatments.

11. The computer-implemented method of claim 9, further comprising:
storing the suggested treatment to a database.

12. A non-transitory computer program product comprising instructions which, when executed by a computer, cause the computer to perform the method of claim 1.

13. A computer-implemented apparatus for characterizing a lesion in a blood vessel depicted in a radiography image, the computer-implemented apparatus comprising:
an identifying unit configured to identify a void in the radiography image, the void being representative of a presence of the lesion; and
at least one processor configured to cause the computer-implemented apparatus to,
determine a distance from the identified void to a region of interest in the radiography image,
determine a size of a nidus located between an artery and a vein,
determine a presence of a deep venous drainage in the radiography image, and
determine a grading of the lesion based on at least one of the determined distance of the void to the region of interest, the size of the nidus or the presence of the deep venous drainage.

14. The computer-implemented apparatus of claim 13, further comprising:
an execution unit.

15. A system for characterizing a lesion in a blood vessel depicted in a radiography image, comprising:
the computer-implemented apparatus of claim 13.

16. The computer-implemented method of claim 2, wherein the trained artificial intelligence model is a Residual Network.

17. The computer-implemented method of claim 4, wherein the localizing comprises determining coordinates of the lesion.

18. The computer-implemented method of claim 8, wherein the grading system is a Spetzler-Martin grading system.

19. The computer-implemented method of claim 9, further comprising:
suggesting the treatment for the lesion further based at least in part on one or more of an age of a patient, country specific guidelines for treating the lesion or a parameter that is used to determine the grading.
